(19) United States
(12) Patent Application Publication
(10) Pub. No.: US 2015/0230694 A1
(43) Pub. Date: Aug. 20, 2015

(54) GAS TIGHT GUIDE FOR BRONCHOSCOPE AND RELATED DEVICES

(71) Applicant: Munson Labs, Colton, WA (US)

(72) Inventor: Roland Munson, Colton, WA (US)

(73) Assignee: Munson Labs, Colton, WA (US)

(21) Appl. No.: 14/183,345

(22) Filed: Feb. 18, 2014

(51) Int. Cl. A61B 1/00; A61M 16/12; A61M 16/10; A61M 16/04; A61B 1/267

(52) U.S. Cl. CPC A61B 1/00154; A61M 16/0488; A61B 1/00128; A61B 1/2676; A61B 1/00137; A61M 16/104; A61M 16/12

(57) ABSTRACT

Gas tight guides for use in veterinary colonoscopy and bronchoscopy procedures which permit the effective sealing of animal airways and anal orifices during endoscopic procedures.

### BACKGROUND OF THE INVENTION

Bronchoscopy allows for the evaluation of the trachea, carina, and bronchi. General anesthesia is required for bronchoscopy in order to suppress coughing, laryngospasm, and to allow examination of the airway without inducing trauma.

The standard procedure in veterinary medicine involves disconnecting the anesthetized patient from oxygen/anesthesia and then inserting the flexible endoscope down the patient's airway. When the patient's blood oxygen saturation falls to below 80%, the endoscope is removed and the oxygen/anesthesia is reconnected to the patient.

After the patient's blood oxygen saturation level has recovered, the whole process of disconnecting the oxygen/anesthesia and reinserting the endoscope is recommenced. These alternating operations continue until the exam is completed. Oxygen can be supplied through the endoscope's biopsy channel if the endoscope is so equipped, which may allow extension of the exam time from seconds to minutes. But, excessive oxygen flow to the patient can result in rupture of alveoli or even of the pneumothorax.

The described invention allows a complete exam to be performed while maintaining blood oxygen saturation levels and without having to remove the endoscope and interrupt the exam.

The inventions disclosed herein pertain to the field of veterinary medical devices. More particularly, the inventions pertain to novel devices and methods for assisting with bronchoscopy, colonoscopy and related procedures in small animals.

Veterinary bronchoscopy and endoscopy procedures are frequently done in veterinary practices, clinics and hospitals. The standard procedure in veterinary medicine involves disconnecting the anesthetized patient from oxygen/anesthesia and then inserting the flexible endoscope down the patient's airway. When the patient's blood oxygen saturation falls to below 80%, the endoscope is removed and the oxygen/anesthesia is reconnected to the patient.

After the patient's blood oxygen saturation level has recovered, the whole process of disconnecting the oxygen/anesthesia and reinserting the endoscope is recommenced. These alternating operations continue until the exam is completed. Oxygen can be supplied through the endoscope's biopsy channel if the endoscope is so equipped, which may allow extension of the exam time from seconds to minutes. But, excessive oxygen flow to the patient can result in rupture of alveoli or even of the pneumothorax.

Limitations of the current art include the inability to perform one continuous exam and the inability to remove multiple foreign bodies during an exam. The current state of the art procedure is time-consuming and introduces risk, when having to extubate and then re-intubate the animal repeatedly during the exam.

In existing procedures of veterinary colonoscopy in small animals, the anal sphincter is manually held closed by a veterinary technician assisting with the procedure. The technician will use two hands and 4-6 fingers to pinch the anal sphincter closed. This prevents the air from exiting the orifice, but requires human assistance during the entire procedure.

In these cases, the use of a human for the purpose of ensuring air-tightness is expensive and very tiring for the person. Another drawback of the current process is the availability of additional personnel to be present during the entire procedure.

Current methods for assisting with air-tightness during scoping procedures in humans include inflated balloon devices that close off the airway during the procedure. However, such devices are complex and therefore too expensive to be commercially practical in a veterinary setting.

Using the invention proposed herein in one embodiment, the personnel required to conduct a typical veterinary colonoscopy procedure in small animals is reduced by one.

The foregoing and other objectives, features, and advantages of the invention will be more readily understood upon consideration of the following detailed description of the invention taken in conjunction with the accompanying drawings.

### SUMMARY OF THE INVENTION

A fitting body defining first, second, and third ports, the first and second ports being coaxial and on opposite ends of the fitting body, the third port extending laterally relative to the first and second ports, the first port having a diameter sufficient to receive a proximal end of an endotracheal tube for introduction of the endotracheal tube into a subject's trachea, the second port including a membrane-holding portion and being configured to receive a longitudinal object intended to fit into the endotracheal tube and then into a subject's trachea, and the third port having a diameter sufficient to receive a gas-delivering conduit for delivery of gas to the subject; an elastomeric membrane mounted to the membrane-holding portion such that the membrane is circumferentially sealed to the membrane-holding portion, the membrane defining an orifice having a diameter sufficient to pass, with stretching of the membrane, the longitudinal object from outside to inside the subject's trachea, the stretching of the membrane providing a gas-tight seal of the membrane to the longitudinal object.

### BRIEF DESCRIPTION OF THE DRAWINGS

FIG. 1 is a longitudinal view of one embodiment of the guide disclosed herein with endoscope, endotracheal tube, and oxygen and anesthesia connected.

FIG. 2 is a longitudinal view of one embodiment of the guide disclosed herein.

FIG. 3 is a top view of one embodiment of the guide disclosed herein.

FIG. 4 is a top view of a cap in accordance with one embodiment of the guide disclosed herein.

FIG. 5 is a side cross sectional view of a head element in accordance with one embodiment of the guide disclosed herein.

FIG. 6 is a top view of a diaphragm in accordance with one embodiment of the guide disclosed herein.

FIG. 7 is a side cross sectional view of one embodiment of the guide disclosed herein.

### DETAILED DESCRIPTION

The proposed Gas-Tight Guide for Bronchoscope and Related Procedures are devices and methods for sealing an orifice during veterinary scoping procedures, which increases efficiency and quality of the procedures. This solution employs novel tool design and associated methods for use. The proposed devices and methods are intended for use primarily in small animal scoping procedures; however, the use in other areas of medicine is not excluded.

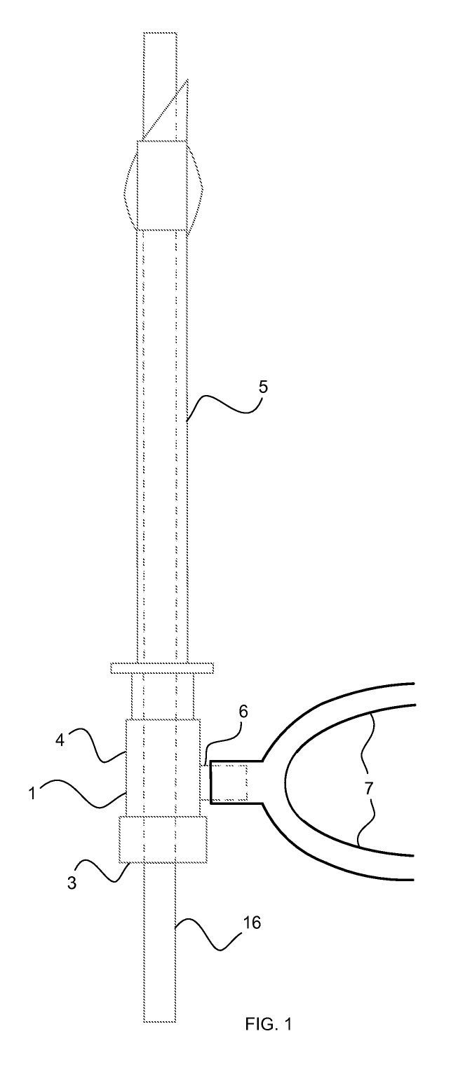

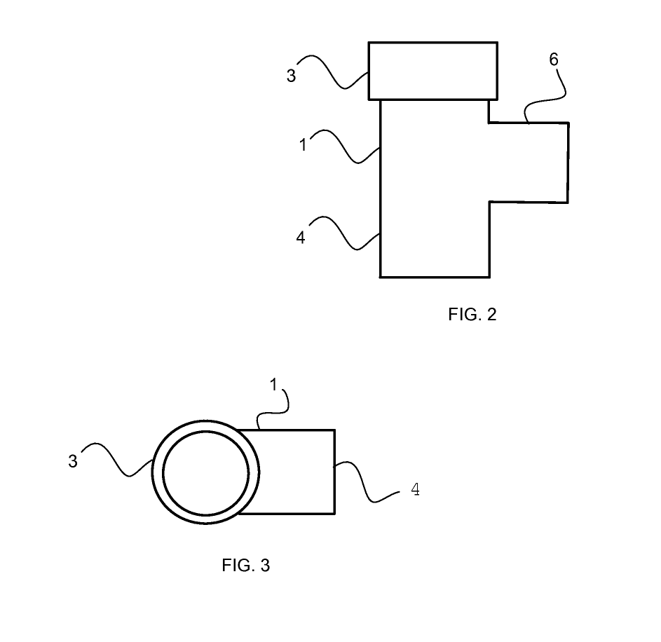

As shown in FIGS. 1-3, the proposed bronchoscopy gas-tight guide 1 device is a hand-held tool which allows for bronchoscopy while administering oxygen/anesthesia and maintaining oxygen saturation throughout the bronchoscopy procedure. It may be configured as a T. It has attachments for the oxygen/anesthesia supply tubes 7 coming from the oxygen/anesthesia machine, and for the fitting from the endotracheal tube 5 in the patient. The oxygen/anesthesia connection port 6 holds the hose 7 from the oxygen/anesthesia machine, and the endotracheal tube connection port 4 holds the fitting from the patient's endotracheal tube 5. The endoscope connection port 3 accepts a diaphragm 11 with a small hole 8 fitting to the diameter of the endoscope 16.

The diaphragm 11 as shown in FIG. 6 comprises a membrane 17 which may be preferably made of an elastomeric membrane material which is preferably thin and may be placed under tension. The membrane 17 is mounted to a membrane-holding portion 18, such as a rigid or semi-rigid frame. The frame may be made of stiffer or thicker elastomeric material which enables the diaphragm to circumferentially seal a port into which it is inserted. The membrane defines an orifice 8 having a diameter sufficient to pass, with stretching of the membrane, the endoscope 16 from outside to inside the subject's trachea, The stretching of the membrane provides a gas-tight seal of the membrane to the endoscope. The endoscope port 3 may further comprise a threaded ring (not shown) that engages the second port to seal the diaphragm circumferentially to the second port. The second port may also include a land which corresponds to a land on the threaded ring. The diaphragm may seal the port when it is placed between lands.

The material used for making the guide may be any inert, rigid biocompatible plastic (polymeric) material such as Nylon, Delrin, or Polypropylene. The guide may take configurations other than a T configuration, so long as the guide preferably comprises three ports, two of which are approximately coaxial.

In an exemplary use, the process to install the bronchoscopy gas-tight guide 1 can be described as follows:

The anesthesia/oxygen supply tube 7 is disconnected from the endotracheal tube 5 placed into the patient.

The bronchoscopy gas-tight guide 1 is connected to the endotracheal tube 5 and the anesthesia/oxygen supply tube 7 is connected to the bronchoscopy gas-tight guide 1. The tube 5 is connected to the port 4 and the oxygen supply tube 7 is connected to port 6. The endoscope inserted into the port 3.

The endoscope is then inserted into the bronchoscopy gas-tight guide 1 to start the bronchoscopy exam.

When the bronchoscopy exam is completed, the endoscope is removed from the bronchoscopy gas-tight guide 1.

Then the bronchoscopy gas-tight guide 1 is disconnected from the anesthesia/oxygen supply tube 7 and the endotracheal tube 5.

The anesthesia/oxygen supply tube 7 is reconnected to the endotracheal tube 5 to complete the procedure.

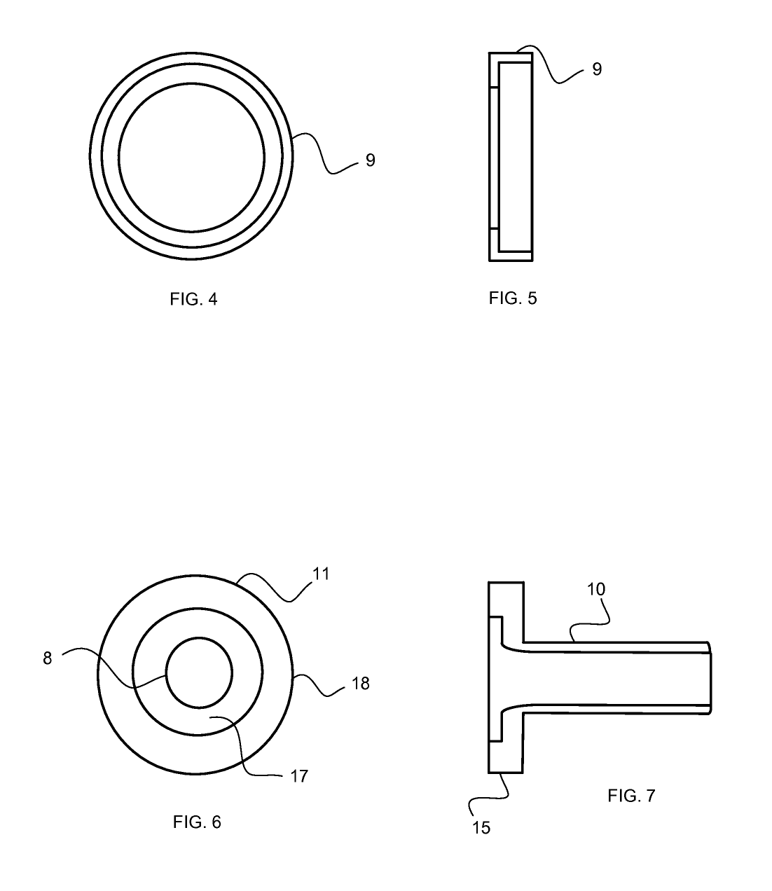

In another implementation, a similar version of the gas-tight guide can be used to perform a colonoscopy exam. As shown in FIGS. 4-5, the proposed colonoscopy gas-tight guide assembly device is a hand-held tool which allows for colonoscopy while ensuring an air-tight orifice for the endoscope to maneuver. The colonoscopy gas-tight guide post 10 has a head element 15 which is press fit into the colonoscopy gas-tight guide cap 9. The colonoscopy gas-tight guide cap 9 holds the flexible diaphragm 11, which is pressed between the head element 15 and the cap 9. The diaphragm 11 has a small hole fitting to the endoscope diameter.

A description of the colonoscopy exam process using the colonoscopy gas-tight guide assembly is as follows:

A diaphragm 11, which can be configured as described above with respect to the endotracheal device, is inserted into the cap 9, and the cap 9 is press fit onto the head element 15. The post 10 is then lubricated and inserted into the anal orifice of the patient.

The endoscope is inserted through the cap 9, the diaphragm 11 via the hole 8 in the membrane 17, and the post 10 on the colonoscopy gas-tight guide assembly to start the colonoscopy exam. The endoscopist applies gentle pressure to keep the colonoscopy gas-tight guide in position throughout the exam.

At the completion of the colonoscopy exam, the endoscopist slides the colonoscopy gas-tight guide assembly out of the anal orifice, and up the working end of the endoscope to complete the last few centimeters of the exam.

With the removal of the endoscope from the patient, the colonoscopy gas-tight guide assembly can be removed from the endoscope.

One of the novel outcomes of using the proposed gas-tight guide tools and associated methodology is a greatly improved efficiency of the scoping procedure. The proposed gas-tight guide tools enable reduced manpower requirements, and in turn, reduced cost of procedure. In the case of bronchoscopy procedures, they also reduce risk for the animal in comparison to the current standard. In addition, the user considers the tools and method described herein to be much easier to use than any current solutions.

In another example, the devices and method described herein could be optimized and scaled for use in a broader segment of animals and procedures.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A fitting body defining first, second, and third ports, the first and second ports being coaxial and on opposite ends of the fitting body, the third port extending laterally relative to the first and second ports, the first port having a diameter sufficient to receive a proximal end of an endotracheal tube for introduction of the endotracheal tube into a subject's trachea, the second port including a membrane-holding portion and being configured to receive a longitudinal object intended to fit into the endotracheal tube and then into a subject's trachea, and the third port having a diameter sufficient to receive a gas-delivering conduit for delivery of gas to the subject;
an elastomeric membrane mounted to the membrane-holding portion such that the membrane is circumferentially sealed to the membrane-holding portion, the membrane defining an orifice having a diameter sufficient to pass, with stretching of the membrane, the longitudinal object from outside to inside the subject's trachea, the stretching of the membrane providing a gas-tight seal of the membrane to the longitudinal object.

2. The device of claim I, wherein the longitudinal object is an endoscope.

3. The device of claim I, wherein the second port further comprises a threaded ring that engages the second port to seal the membrane circumferentially to the second port.

4. The device of claim 3, wherein the second port includes a respective land; the threaded ring includes a respective land; and the membrane is circumferentially sealed to the second port whenever the membrane is placed between the lands and the ring is snugly threaded onto the second port.

5. A bronchoscopic procedure, comprising:
placing a guide device external to and relative to a subject's mouth, the guide device comprising a fitting body defining first, second, and third ports, the first and second ports being coaxial and on opposite ends of the fitting body, the third port extending laterally relative to the first and second ports, the first port having a diameter sufficient to receive a proximal end of an endotracheal tube for introduction of the endotracheal tube into the subject's trachea, the second port including a membrane-holding portion and being configured to receive an endoscope intended to fit into the endotracheal tube and then into the subject's trachea, and the third port having a diameter sufficient to receive a gas-delivering conduit for delivery of respiratory and anesthetic gas to the subject, the second port including an elastomeric membrane mounted to the membrane-holding portion such that the membrane is circumferentially sealed to the membrane-holding portion, the membrane defining an orifice having a diameter sufficient to pass, with stretching of the membrane, the endoscope from outside to inside the subject's trachea, the stretching of the membrane providing a gas-tight seal of the membrane to the endoscope; and
inserting an endoscope into the subject's trachea via the orifice in the membrane holder as the membrane around the orifice stretches to accommodate passage of the endoscope and form a gas-tight seal around the endoscope.
